(19) United States
(12) Patent Application Publication
(10) Pub. No.: US 2001/0041902 A1
(43) Pub. Date: Nov. 15, 2001

(54) ANASTOMOTIC METHODS AND DEVICES FOR PLACING A TARGET VESSEL IN FLUID COMMUNICATION WITH A SOURCE OF BLOOD

(75) Inventors: KEKE J. LEPULU, REDWOOD CITY, CA (US); DEAN F. CARSON, MOUNTAIN VIEW, CA (US); DAVID H. COLE, SAN MATEO, CA (US); A. ADAM SHARKAWY, UNION CITY, CA (US); STEVE L. OLSON, SUNNYVALE, CA (US); SAMUEL T. CREWS, REDWOOD CITY, CA (US); MICHAEL L. REO, REDWOOD CITY, CA (US); DARIN C. GITTINGS, SUNNYVALE, CA (US); MARK J. FOLEY, MENLO PARK, CA (US)

Correspondence Address: Michael J. Lynch, Hoekendijk & Lynch LLP, P.O. Box 4787, Burlingame, CA, 94011-4787, US

(73) Assignee: Michael J. Lynch

(21) Appl. No.: 09/393,130

(22) Filed: Sep. 10, 1999

Related U.S. Application Data
(63) Continuation-in-part of application No. 09/023,492, filed on Feb. 13, 1998.

(51) Int. Cl. A61B 17/00

(52) U.S. Cl. 606/153

(57) ABSTRACT

Anastomotic methods and devices for placing a target vessel in fluid communication with a target vessel. A conduit includes an attachment portion adapted to be secured to a target vessel in fluid communication with the lumen of the vessel. The target vessel wall is sandwiched between first and second components of the attachment portion to provide both a secure and sealed connection. One component is placed in the vessel lumen against the interior surface of the wall and has an outlet that directs blood into the target vessel. This component is elongated, e.g., elliptical or rectangular, such that a minimum amount of material is present at the outlet. This results in the outlet having a diameter that substantially maintains much of the cross-sectional area of the native target vessel. The attachment portion does not significantly occlude the target vessel lumen, is secured to the vessel wall in non-penetrating fashion, allows a small incision in the vessel wall, and provides a fluid-tight seal around the attachment site. The conduit may comprise tissue, synthetic material, etc., and the attachment portion may be configured for attachment to a native graft vessel, such as an internal mammary artery.

### CROSS-REFERENCE TO RELATED APPLICATIONS

This application is a continuation-in-part of application Ser. No. 09/023,492, filed on Feb. 13, 1998 and entitled “Methods and Devices Providing Transmyocardial Blood Flow to the Arterial Vascular System of the Heart,” the entire subject matter of which is incorporated herein by reference. This application is also a continuation-in-part of application Ser. No. 09/232,103, filed on Jan. 15, 1999 and entitled “Methods and Devices for Forming Vascular Anastomoses,” as well as application Ser. No. 09/232,062, filed on Jan. 15, 1999 and entitled “Methods and Devices For Bypassing an Obstructed Target Vessel by Placing the Vessel in Communication with a Heart Chamber Containing Blood,” the entire subject matter of both applications being incorporated herein by reference.

### BACKGROUND OF THE INVENTION

1. Field of the Invention

The invention relates to placing a target vessel in fluid communication with a source of blood, and more particularly anastomotic methods and devices for establishing communication between the vessel and the blood source.

2. Description of Related Art

Despite the considerable advances that have been realized in cardiology and cardiovascular surgery, heart disease remains the leading cause of death throughout much of the world. Coronary artery disease, or arteriosclerosis, is the single leading cause of death in the United States today. As a result, those in the cardiovascular field continue to search for new and improved treatments.

Coronary artery disease is currently treated by interventional procedures such as percutaneous transluminal coronary angioplasty (PTCA), coronary stenting and atherectomy, as well as surgical procedures including coronary artery bypass grafting (CABG). The goal of these procedures is to reestablish or improve blood flow through occluded (or partially occluded) coronary arteries, and is accomplished, for example, by enlarging the blood flow lumen of the artery or forming a bypass that allows blood to circumvent the occlusion. What procedure(s) is used typically depends on the severity and location of the blockage(s). When successful, these procedures restore blood flow to myocardial tissue that had not been sufficiently perfused due to the occlusion.

The improvement and refinement of existing treatments and the search for new treatments are indicative of the significant effort that continues to be expended in order to develop better and more efficient ways of revascularizing the heart. An alternative, recently proposed treatment places the target vessel in fluid communication with a heart chamber containing blood, for example, the left ventricle. Blood flows from the ventricle into a conduit that is in fluid communication with the target vessel. Some of the challenges associated with these procedures include delivering and deploying the conduit in the patient's body, properly positioning the conduit with respect to the heart chamber and the target vessel, and obtaining beneficial flow characteristics through the target vessel. A particularly challenging task that is performed in these procedures is securing the conduit to the target vessel, which in the case of a coronary artery has a diameter of 1 mm to 4 mm. In a conventional CABG procedure the conduit is secured to the artery by a handsewn distal anastomosis, a very technical and time-consuming procedure.

Accordingly, there remains a need in the art for improved methods and devices for making anastomotic connections between hollow bodies such as coronary vessels quickly, easily and in a repeatable manner to carry out cardiac revascularization procedures.

### SUMMARY OF THE INVENTION

One embodiment of the invention provides a method for delivering blood from a heart chamber containing blood to a target vessel of a patient's vascular system. The method includes steps of providing a conduit having a lumen adapted to be placed in fluid communication with a heart chamber containing blood and an attachment portion adapted to be secured to a target vessel so as to communicate with the lumen of the target vessel, placing the conduit in fluid communication with the heart chamber, positioning a first component of the conduit attachment portion in the target vessel lumen adjacent a first area of the vessel wall, and positioning a second component of the conduit attachment portion adjacent another area of the wall, capturing the target vessel wall between the first and second components to secure the conduit to the target vessel, and delivering blood from the heart chamber into the conduit during at least one phase of the heart cycle.

Another method is for securing a conduit to a target vessel of a patient's vascular system and includes steps of providing a conduit having a lumen and an attachment portion adapted to be secured to a target vessel so that the conduit lumen is in fluid communication with the target vessel lumen, wherein the attachment portion includes first and second securing components, placing the first securing component at least partially with the target vessel lumen and positioning the first securing component adjacent one surface of the target vessel wall, placing the second securing component adjacent another surface of the target vessel wall, applying a desired amount of force to the target vessel wall without the first or second component penetrating the tissue of the target vessel wall, thereby securing the conduit to the target vessel.

A method for securing a native artery to a target vessel of a patient's vascular system includes steps of providing an attachment mechanism configured to be secured to a target vessel, the attachment mechanism having a lumen adapted to be placed in fluid communication with the target vessel lumen, wherein the attachment mechanism includes first and second securing components, placing one of the securing components at least partially within the lumen of the target vessel adjacent a surface of the target vessel wall, preparing the native artery to provide an exposed portion for securing the artery to the target vessel, coupling the other securing component to the exposed portion of the native artery and positioning the other securing component adjacent another surface of the target vessel wall, and sandwiching the target vessel wall between the securing components to secure the native artery to the target vessel.

A device constructed according to the invention for securing a conduit to a target vessel so that the conduit and the vessel are in fluid communication includes first and second securing components configured to engage different areas of the wall of a target vessel to provide a secure attachment, and at least one of the components has a length and a width, the length being defined generally along the axis of the target vessel when the device is positioned in the target vessel. The length of the at least one securing component is greater than the width of the at least one securing component.

Another device constructed according to the invention includes first and second securing components configured to engage different areas of the wall of a target vessel to provide a secure attachment, at least one of the components having a length and a width, the length being defined generally along the axis of the target vessel when the device is positioned in the target vessel. One of the first and second securing components is configured to directly engage an opening in the wall of a target vessel and is sized for a particular size range of target vessels, and the one securing component has an outlet with a cross-sectional area of at least 50% of the cross-sectional area of the target vessels in the particular size range.

### BRIEF DESCRIPTION OF THE DRAWING FIGURES

The invention will be better understood from the following detailed description of preferred embodiments thereof, taken in conjunction with the accompanying drawing figures, wherein:

FIG. 1 is a perspective view of a conduit constructed according to one embodiment of the invention for placing a source of blood in fluid communication with a target vessel, the conduit being shown in a target vessel securing position;

FIG. 2 is a perspective view of the conduit shown in FIG. 1, the conduit being shown in a target vessel release position;

FIG. 3 is an exploded view of a conduit constructed according to another embodiment of the invention;

FIG. 4 is a perspective view of the conduit shown in FIG. 1 with the attachment portion partially disposed in the target vessel;

FIG. 5 is a perspective view of the conduit shown in FIG. 4 after the attachment portion has been moved to a vessel securing position

FIG. 6 is a perspective view of the conduit shown in FIG. 5 in fluid communication with a heart chamber containing blood;

FIGS. 7A and 7B are perspective views respectively showing a conduit constructed according to another embodiment of the invention both out of and in communication with a heart chamber containing blood;

FIGS. 8-10 are perspective views of conduits constructed according to other embodiments of the invention for placing a source of blood in fluid communication with a target vessel;

FIGS. 11-12 are perspective views of conduits constructed according to still other embodiments of the invention for placing a source of blood in fluid communication with a target vessel;

FIG. 13 is a sectional view of a conduit constructed according to another embodiment of the invention for placing a source of blood in fluid communication with a target vessel;

FIGS. 14A-14B are elevation views showing, respectively, a conduit constructed according to another embodiment of the invention in target vessel securing and release positions;

FIGS. 15A-15B are elevation views showing a conduit constructed according to another embodiment of the invention in, respectively, target vessel securing and release positions;

FIG. 16 is a perspective view of a conduit constructed according to another embodiment of the invention;

FIG. 17 is a sectional view of a conduit constructed according to another embodiment of the invention;

FIG. 18A is a perspective view of a conduit constructed according to another embodiment of the invention;

FIG. 18B is a plan view of the conduit shown in FIG. 18A;

FIGS. 19 and 20 are sectional views of conduits constructed according to other embodiments of the invention; and

FIGS. 21A and 21B are perspective views showing the conduit shown in FIGS. 1 and 2 mounted on a delivery device constructed according to another embodiment of the invention, the conduit being shown, respectively, in target vessel securing and release positions.

### DETAILED DESCRIPTION OF PREFERRED EMBODIMENTS

One aspect of the present invention provides a conduit that is placed in a patient's body to establish a flow path between a source of blood and a target vessel, as well as methods and device for deploying the conduit. In a broader aspect the invention provides methods and devices for securing and sealing a conduit to a target vessel.

In a preferred embodiment, the conduit is coupled to source of blood, e.g., a heart chamber containing oxygenated blood, and a target vessel, e.g., a coronary vessel (artery or vein). It will be recognized, however, that the invention may be used to form a blood flow path between other hollow body structures. Also, as used herein, source of blood refers to any blood-containing structure, while oxygenated blood refers to blood containing some level of oxygen.

The lumen of the target vessel may be partially or completely obstructed by an occlusion and the conduit placed to form a flow path that bypasses the occlusion. Alternatively or additionally, the conduit may be used to create a supplemental blood flow path that feeds into the target vessel to augment blood flow (native or other) already present in the vessel.

The conduit of the invention may be configured in various manners. In its most preferred form, the conduit includes an attachment portion configured to be secured to the target vessel wall and form an anastomotic connection between the vessel and the conduit. The attachment portion may secure to the target vessel wall by various means that achieve a secure attachment and hemostasis, preferably by a tight seal against the tissue of the vessel wall. The attachment portion also moves between target vessel securing and release positions to allow easy, low profile introduction into the vessel. For use in a ventricular bypass procedure, the conduit has a first portion configured to be placed in the myocardium and a second portion placed in fluid communication with a coronary vessel.

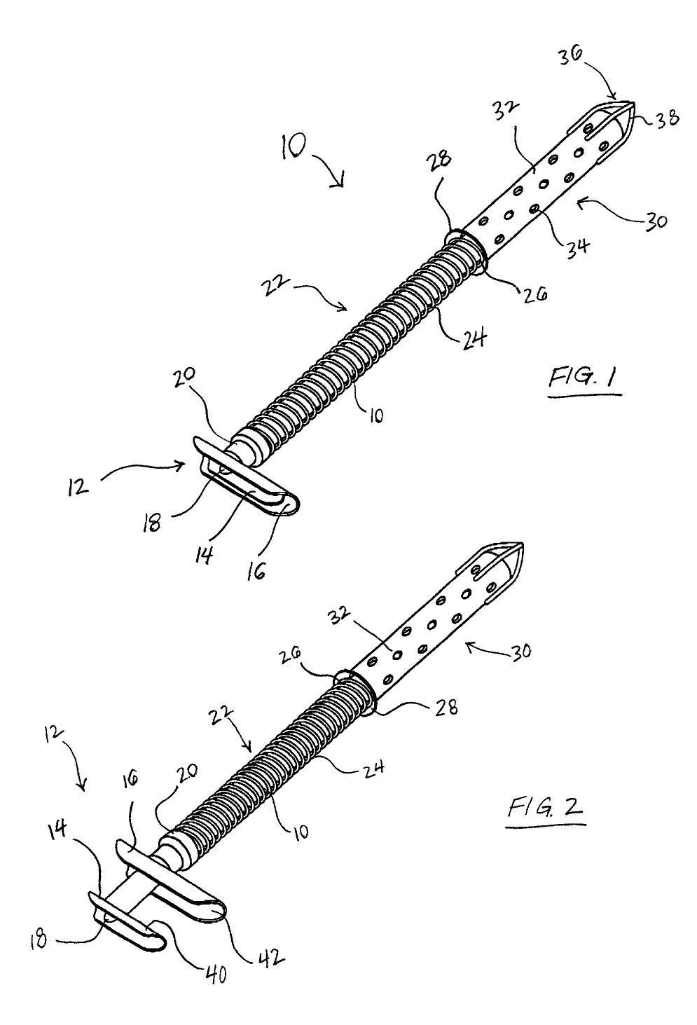

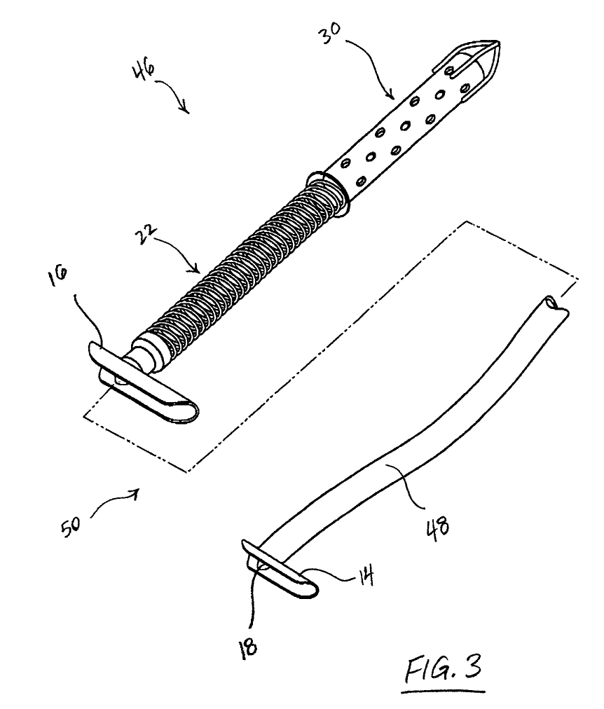

FIGS. 1 and 2 show a conduit 10 constructed according to one embodiment of the invention. The conduit 10 is in the form of an elongated tubular length of vascular graft material, either tissue, synthetic, or both. At least one end of the conduit 10 has an attachment portion 12 including a first component 14 movably supporting a second component 16 having a lumen 18. The conduit 10 may be formed integrally with the component 12 or it may be attached thereto by suitable means, for example, suture, fasteners, clamps, clips, etc. The same is true for the component 14 and the proximally located structure to which it is attached via a collar 20. In the illustrated embodiment, the second component 16 of the attachment portion 12 is coupled to a force applying mechanism 22 configured to drive the first and second components 14, 16 toward each other, for example, to the position shown in FIG. 1.

The force applying mechanism 22 shown in FIGS. 1 and 2 (alternatives of which are described below) is constructed to cause a portion of the target vessel wall tissue to be sandwiched or captured between the first and second components 14, 16. Specifically, a coil spring 24 is fixed at one end to the collar 20 and at another end 26 to the flange 28 of a device 30 for communicating with a heart chamber containing blood. The spring 24 drives the components 14, 16 toward each other as shown in FIG. 1. The device 30 comprises a tube 32 with openings 34, and a cage 36 with struts 38 for preventing blockage of the conduit 10. The device 30 is adapted to be positioned in the myocardium and is capable of withstanding myocardial contraction during systole so that the conduit 10 remains open during use. The device 30 may be constructed according to the teachings of co-pending, commonly-owned application Ser. No. 09/304,140, filed on May 3, 1999, and entitled “Methods and Devices for Placing a Conduit in Fluid Communication with a Target Vessel,” the entire subject matter of which application is incorporated herein by reference.

The conduit 10 may comprise a synthetic vascular graft material such as expanded polytetrafluoroethylene (PTFE), polyethylene terephthatlate (Dacron), and polyurethanes, such as Tecoflex, polycarbonate polyurethane—PCPU, such as Biospan (Corethane). Another suitable synthetic material is silicone, such as MED-4850, MED-6640, and MED-gumstock, all commercially available from NuSil Technology of Carpinteria, Calif. The conduit may also be formed of metal or a metallic alloy such as titanium, stainless steel, and nickel titanium. Finally, it should be noted that the conduit could comprise an autologous tissue graft, for instance, a saphenous vein graft harvested from the patient, an allograft or a xenograft. It will also be appreciated that the conduit may comprise any of the aforementioned materials alone or in combination. The conduits also may be provided with means for detecting its position fluoroscopically, e.g., radiopaque markers, which may be desired during or after placement of the conduit in order to allow the user to confirm the position of and blood flow through the conduit.

Similarly, the first and second components 14, 16 of the attachment portion 12 may be formed of any suitable material, such as stainless steel, titanium, nitinol, polymers, or any of the materials listed above or described in the previously incorporated applications with respect to the conduit. Additionally, the components 14, 16 may be coated with or have applied thereto any suitable materials, including those specified above. For example, the first or inner component 12 may be titanium with a silicone coating.

FIG. 1 shows the conduit 10 in a target vessel securing position in which the first and second components 12, 14 are together so as to clamp a vessel wall (not shown in FIGS. 1 or 2). The coil spring 24 is relaxed and forces the components together. In this position the attachment portion 12 provides a secure and sealed connection to the vessel wall, and the first and second securing components 12, 14 preferably have complementarily-shaped surfaces 40, 42, respectively. The first component lumen 18 is, according to the invention, sized to maintain a substantial portion of the cross-sectional area of the target vessel for increased flow. That is, the diameter of the outlet which is defined by lumen 44 is preferably maximized relative to the diameter (or cross-sectional area of the native target vessel). The cross-sectional area of the lumen 40 is at least 50% of the native target vessel area, and preferably 70-80% or higher of the native vessel area. In the preferred and illustrated embodiment, this achieved by forming the inner vessel securing component with an elongated shape having a length greater than its width, and by giving the component a somewhat planar shape to occupy less area in the lumen.

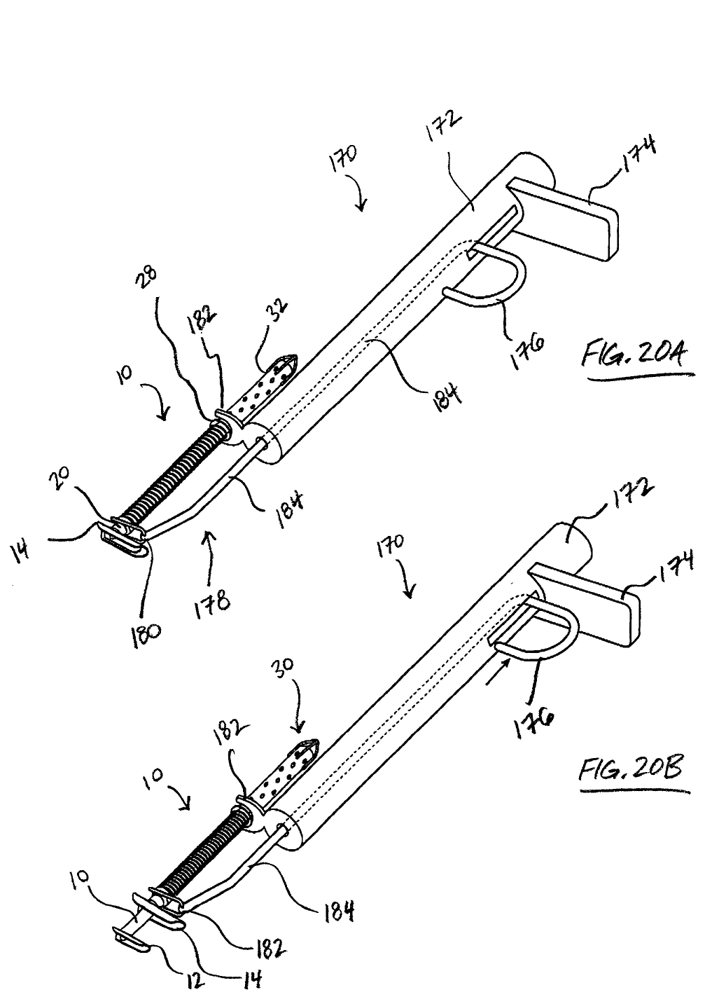

FIG. 2 shows the conduit 10 in a target vessel releasing (or engaging, as it is in this position when first engaged with the vessel wall) position. The first and second components 12, 14 are apart and which allows the components to be manipulated with respect to the target vessel wall. In this position, the coil spring 24 is compressed and must be held until it is time to bring the securing components 12, 14 together. An exemplary delivery device for performing this task is discussed below with respect to FIGS. 21A-21B.

As noted above, the conduit 10 may comprise a tissue graft or a synthetic graft (or a combination of the two). FIG. 3 shows a conduit 46 comprising an autologous vessel 48, such as a saphenous vein graft, secured to an attachment portion 50 that is the same as or similar to the portion 12 above. The conduit 46 may be secured to the attachment portion 50 by suitable means, for example, suture, fasteners, clamps, clips, stents, collagen based adhesives, etc. The attachment portion 50 also could be secured to a native artery such as an internal mammary artery. For instance, the artery could be taken down and the attachment portion 50 secured to an end thereof as disclosed herein. The artery would then be anastomosed to a coronary vessel via the attachment portion 50. The opposite end of the vessel 48 could then be anastomosed to the aorta or another coronary artery.

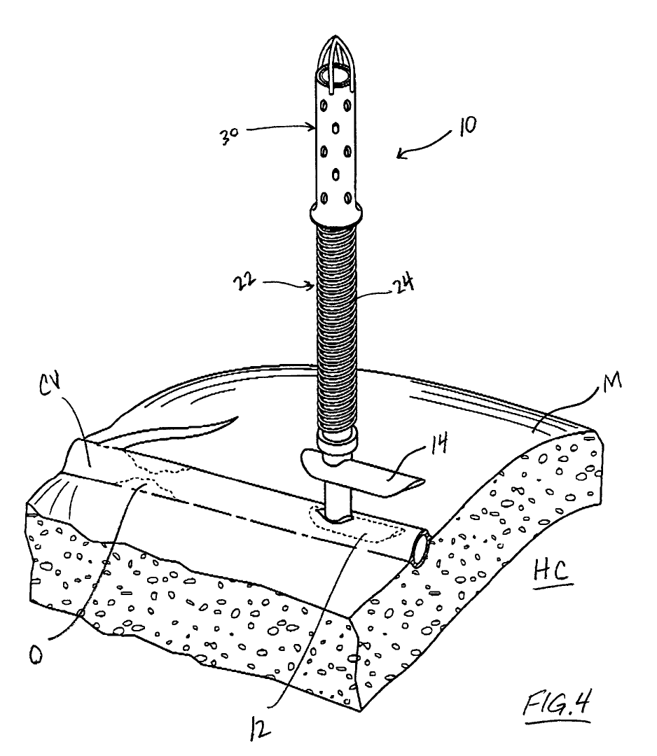

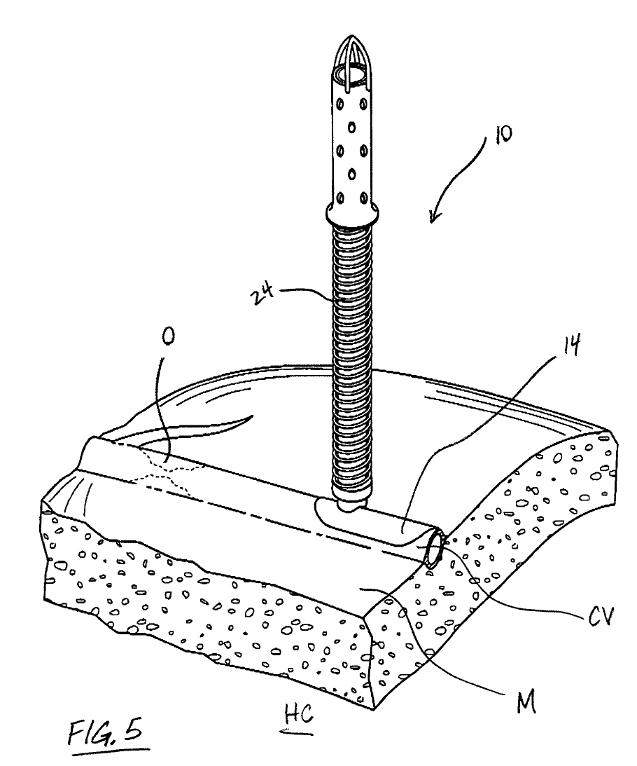

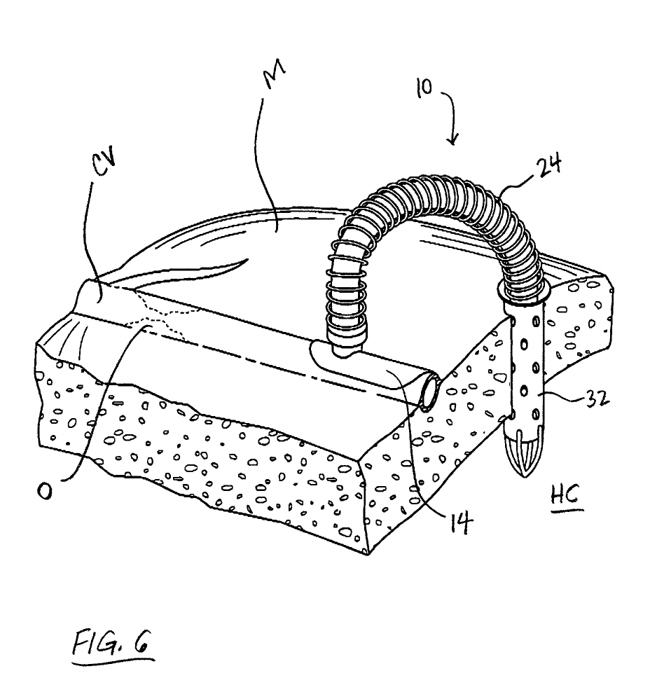

An exemplary use of the conduit 10 shown in FIGS. 1 and 2 will now be described with respect to FIGS. 4-6. The first component 12 of the attachment portion 12 is placed in the lumen of coronary vessel CV containing an occlusion O, and the second component 14 is held in a retracted position by a suitable device (not shown in FIGS. 4-6). FIG. 5 shows the second component 14 moved down against the exterior surface of the vessel wall. FIG. 6 shows the other end of the conduit 10 is placed in fluid communication with a heart chamber HC containing blood.

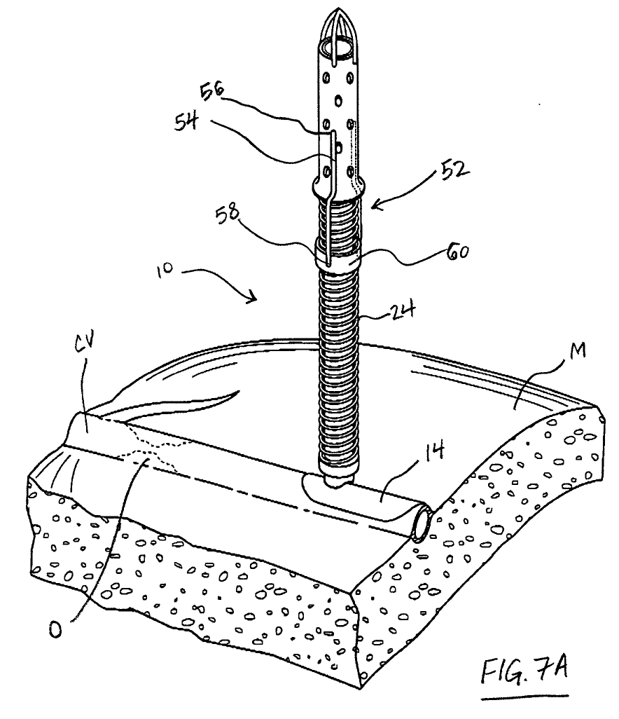

FIGS. 7A-7B show another embodiment of the invention wherein the conduit 10 is provided with a mechanism 52 to control positioning of the conduit body in use. The mechanism includes one or more struts 54 attached at one end 56 to the heart chamber access device 30 and attached at another end 58 to a band 60 fixed to the coil spring 24. FIG. 7A depicts the mechanism 52 prior to the device 30 being placed in the heart chamber. FIG. 7B shows the device 30 in communication with the heart chamber and the mechanism 52 deformed to a final desired position. As can be seen by comparing FIG. 7B and FIG. 6, the mechanism 52 may be used to achieve a lower, flatter conduit profile with respect to the exterior of the myocardium.

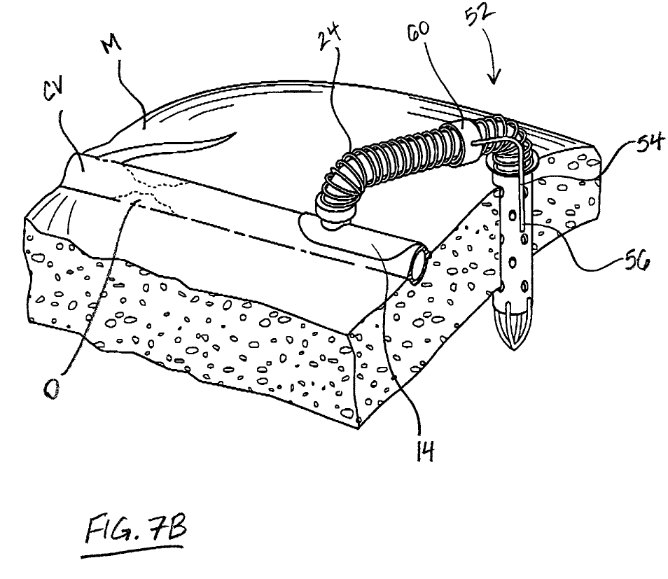

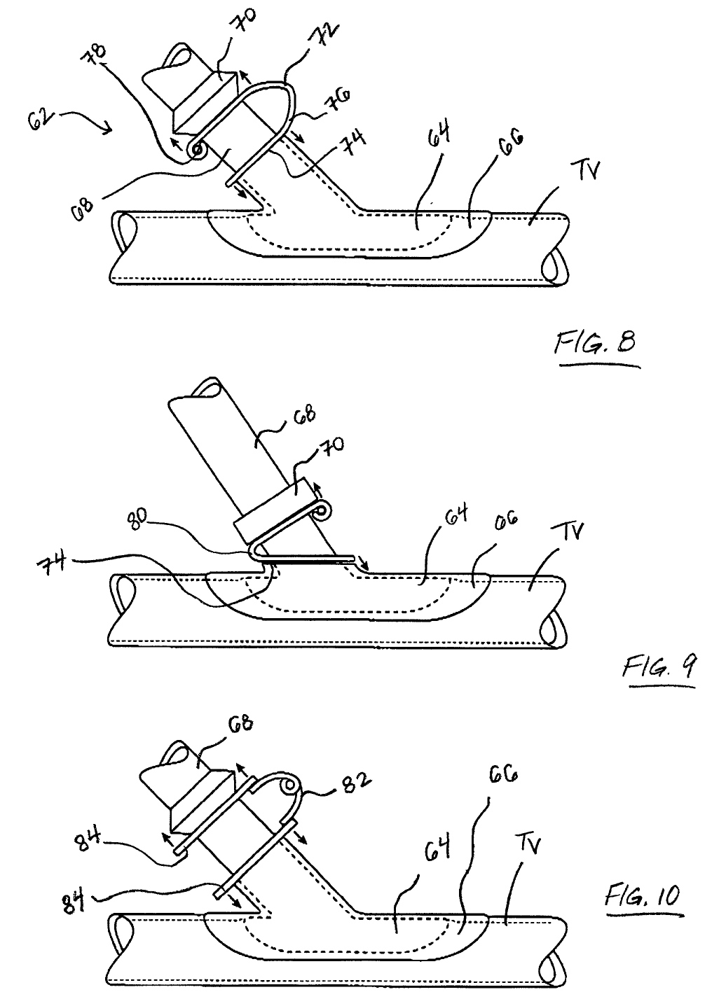

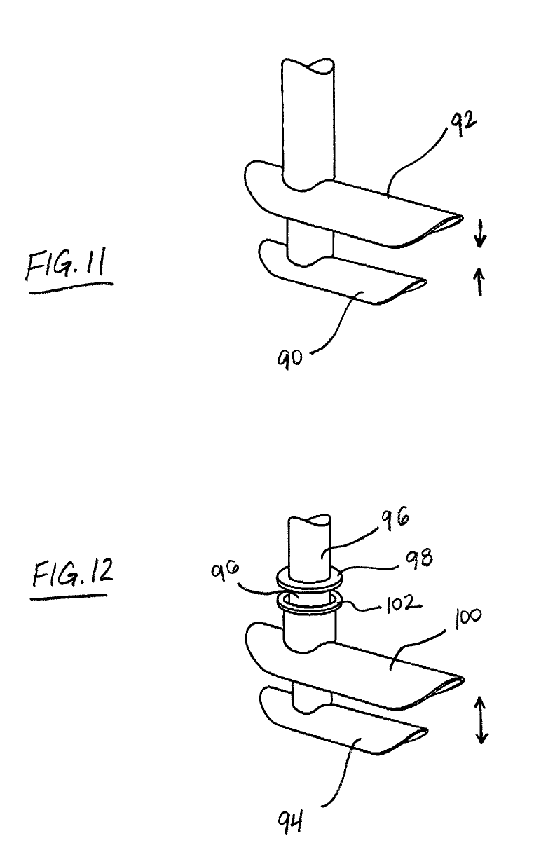

It should be recognized that the coil spring 24 shown in FIGS. 1-2 is only one example of a force applying mechanism. FIGS. 8-10 show other embodiments of the invention wherein alternative springs are used to apply the force used to secure the conduit to the target vessel wall. In FIG. 8 a conduit attachment portion 62 includes first and second components 64, 66 secured to a target vessel TV. The first component 64 communicates with a conduit body 68 having a stop 70. A spring clip 72 is located between the stop 70 and a shoulder 74. The spring has two legs 76 and a pin 78 holds the clip 72 on the conduit body. The resultant force drives the components 64, 66 toward each other to capture the vessel wall. FIG. 9 shows a similar spring clip 80, and FIG. 10 shows a spring 82 disposed between two pieces 84 that engage the first and second components.

Force applying mechanisms that do not utilize springs to impart relative movement to the components may be used as well. FIGS. 11 and 12 shows an embodiment utilizing magnets to secure the target vessel wall. In FIG. 11, a first securing component 90, preferably having a rectangular shape with rounded ends, is formed of magnetic material (or provided with magnetic material), as is a second securing component 92. The poles of the magnets are arranged to attract the components 90, 92 to one another and capture the tissue of the target vessel wall. FIG. 12 shows another embodiment wherein a first securing component 94 is carried by a conduit body 96 which is provided with a magnetic collar 98. A second securing component 100 has a magnetic collar 102, the poles of these magnets being arranged to repel the collars and force the components 94, 100 together.

Another alternative force applying mechanism 104 is shown in FIG. 3 and comprises a resilient member 106 that surrounds first and second securing components 108, 110. The member 106, which may an O-ring, is moved from the position in phantom and forced over a shelf 112 carried by the first securing component to engage the second securing component 110. The resultant force compresses the vessel wall W between the components and also seals the conduit to the target vessel.

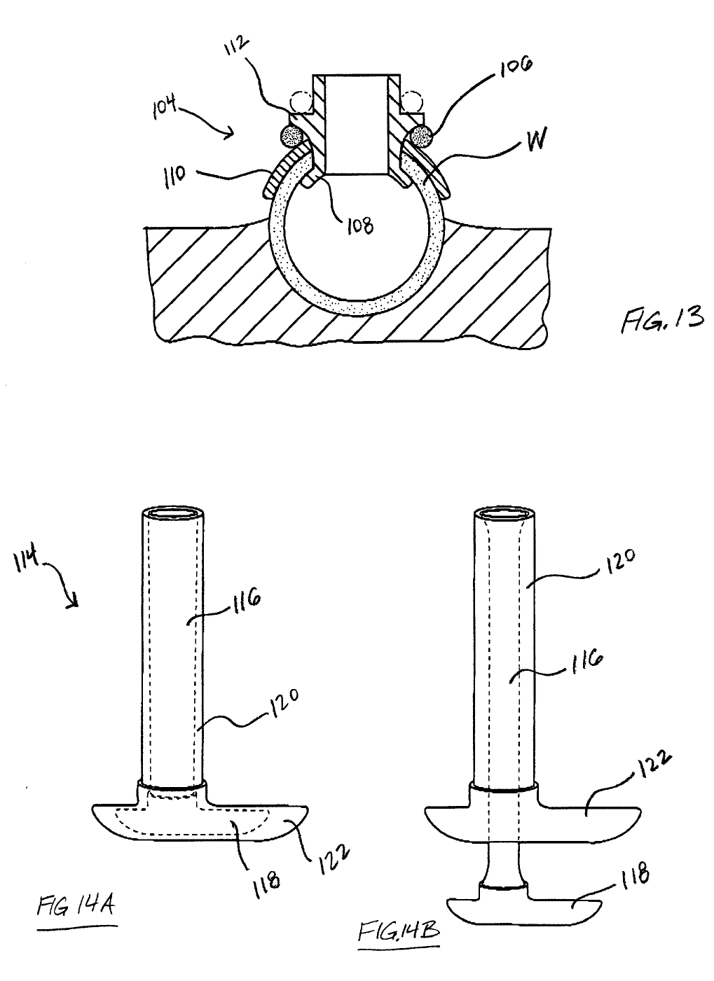

Another alternative force applying mechanism 114 is shown in FIGS. 14A-14B and comprises a resilient conduit 116 supporting a first securing component 118. The conduit 116 is joined to an outer sleeve 120 which supports a second securing component 122. The conduit 116 may be formed of any suitable resilient materials such as silicone. FIG. 14A shows the mechanism 114 at rest with the components 118, 122 forced together. In use, the first securing component 118 is moved to the position shown in FIG. 14B by deforming the conduit 116 and placed in the target vessel. The conduit 116 is then allowed to retract to the position shown in FIG. 14A to capture the vessel wall and provide a fluid-tight seal.

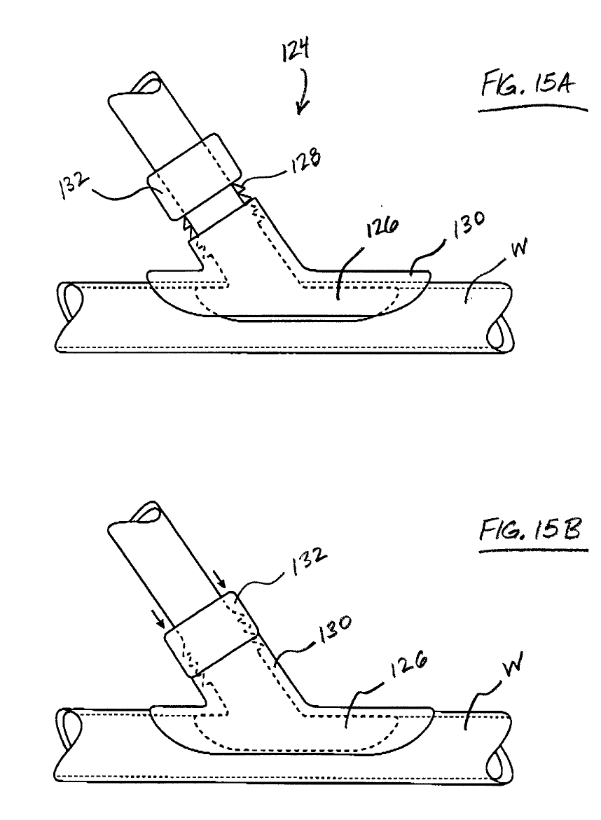

Still another force applying mechanism 124 is shown in FIGS. 15A and 15B. The mechanism 124 comprises a first securing component 126 provided with ratchet teeth 128 on its exterior. A second securing component 130 overlies the vessel wall W and a collar 132 is slid against it to fix the components (FIG. 15B). The collar 132 and/or the second securing component 130 may have recesses adapted to receive the teeth 128, which are preferably configured to prevent separation of the securing components, although they may be constructed to allow separation.

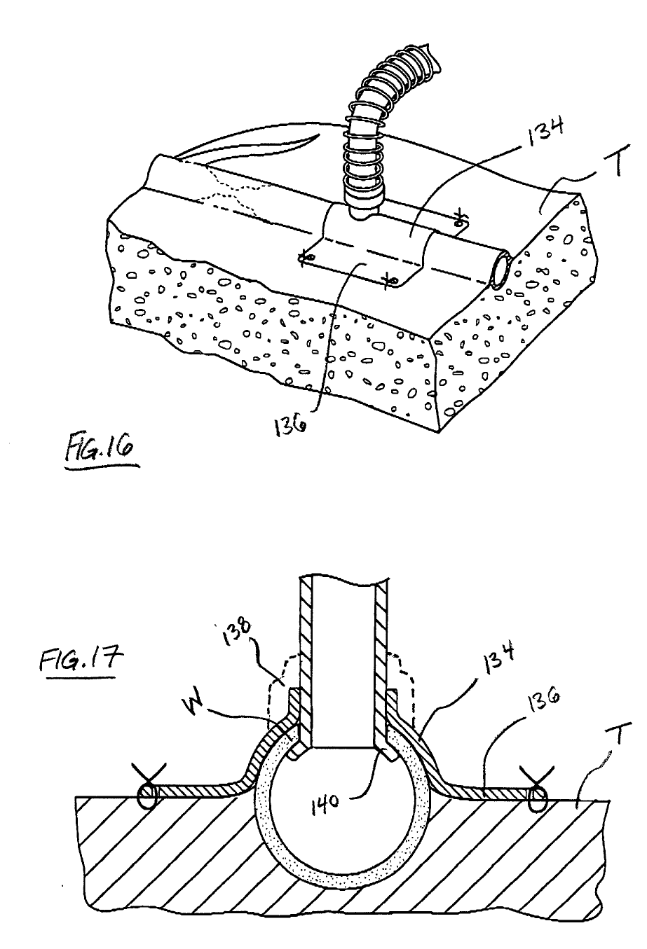

FIG. 16 shows an embodiment wherein the second securing component 134 has a wide configuration to provide portions 136 overlying and supporting the vessel. The portions 136 may be sutured to the tissue, T, as shown. FIG. 17 is a sectional view corresponding to FIG. 16 but schematically illustrating a force applying mechanism at 138 that captures the vessel wall W between the securing components 134, 140.

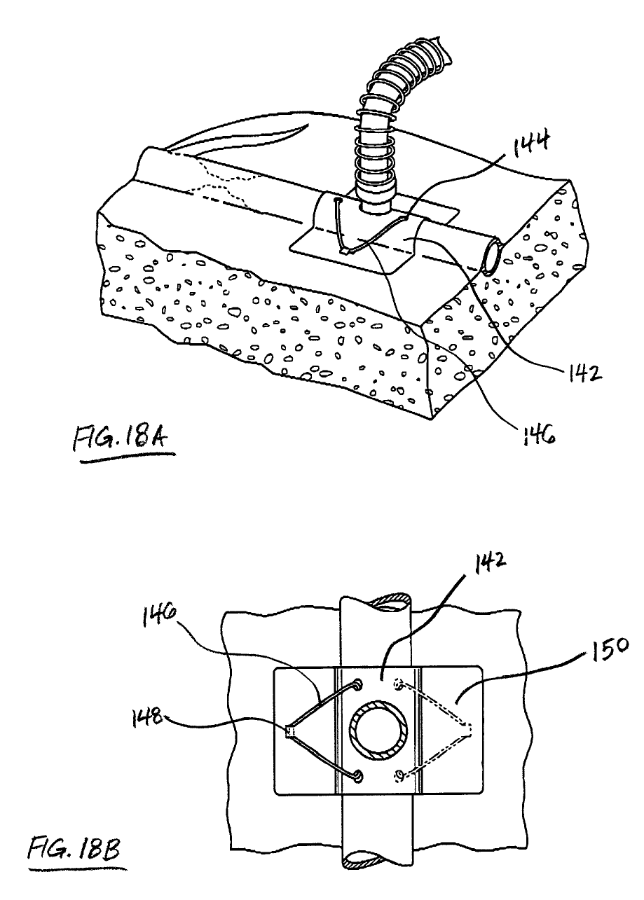

The first and second securing components may be secured to or biased toward each other by lengths of suture, wire, or wire-like material. FIGS. 18A-18b shows an embodiment with a second securing component 142 similar to the component 134 above, but wherein the component 142 has openings 144 through which a cord or wire 146 passes. The cord 146 is secured to a first securing component (not shown) engaged with a surface of the target vessel, and extends upward through the openings 144. The cord 146 is tensioned to force the two securing components toward each other, as shown in the Figures. Any suitable means for retaining the cord 146 in this position may be used, e.g., a hook 148. Also, any suitable number or pieces of cord 146 (or other member) may be used, as represented by the cord 150 in phantom.

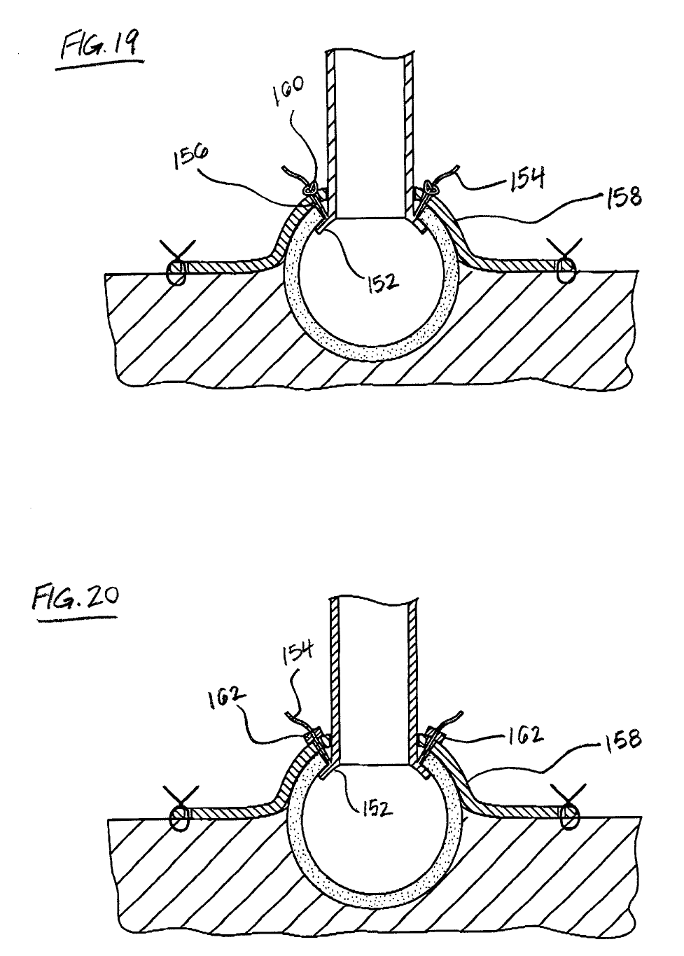

FIG. 19 shows another embodiment in which a first securing component 152 has one or more lengths of suture 154 or other material which extend through openings 156 in a second securing component 158. The suture 154 may be knotted, as shown at 160 in FIG. 20, or it may be secured by a member 162 that is fastened, crushed, crimped or otherwise fixed to the suture 154.

FIGS. 20A-20B show an exemplary delivery device that may be used to support the conduit, and in particular the conduit attachment portion, during engagement of the conduit with the target vessel (and/or the source of blood). The device 170 includes a body 172, a handle 174, and actuator 176, and a conduit supporting section 178. The conduit supporting section 178 includes first and second yokes 180, 182. The first yoke 180 is carried by a shaft 184 controlled by the actuator 176.

FIG. 20A shows the conduit 10 (of FIG. 1) mounted on the device 170 with the yoke 180 engaging the collar 20 and the yoke 182 engaging the body 32 of the heart chamber access device 30, just behind the flange 28. The actuator 176 is in a forward position and the coil spring 24 is relaxed to force the attachment portion 12 to its conduit securing position. FIG. 20B shows the device after the actuator 176 has been moved to a rear position which collapses the coil spring 124 and separates the first and second securing components 12, 14. The first securing component 12 can then be inserted into the lumen and engaged with the interior surface of the vessel wall. The actuator is then used to allow the force of spring 124 to move the components 10, 12 toward each other to capture the target vessel wall as explained above.

The conduit may be constructed differently from the configurations specifically illustrated herein. For example, the conduit could be made according to any of the teachings of co-pending, commonly owned application Ser. No. ______, filed on Sep. 10, 1999 (Attorney Docket No. 010) and entitled “Conduits for Placing a Target Vessel in Fluid Communication With a Source of Blood,” the entire subject matter of which application is incorporated herein by reference.

Moreover, the conduit of the invention may be manufactured by various processes and from various materials; for example, the conduit may be molded (or fabricated from) a material having desired blood interface qualities as well as a desired combination of flexibility and column strength. Manufacturing processes and materials for forming the conduits disclosed herein are disclosed in co-pending, commonly owned application Ser. No. ______, filed on Sep. 10, 1999 (Attorney Docket No. 011) and entitled “Methods and Devices for Manufacturing a Conduit for Use in Placing a Target Vessel in Fluid Communication With a Source of Blood,” the entire subject matter of which application is incorporated herein by reference.

The type of procedure (e.g., open chest, minimally invasive, percutaneous, etc.) that is used to deploy the conduit of the invention may vary depending on the vessels being treated and user preference. As an example, a minimally invasive procedure may be used to deploy the conduit on a beating heart using various devices and methods for stabilizing all or a portion of the heart. Also, the conduits may be coupled to the target vessel other than as specifically shown herein. While several collapsible conduits are illustrated along with exemplary methods for deploying them in a target vessel, it will be appreciated that the invention encompasses securing non-collapsible conduits to the vessel. For instance, the second conduit portion may be a non-collapsible, tubular member that is placed in the target vessel lumen after first dilating the vessel wall, and then is retained by allowing the vessel wall to move back and snugly engage the exterior of the second conduit portion.

It should also be noted that the conduits of the invention may be introduced into a target vessel in various ways. For example, in the illustrated embodiment, the second conduit portion is inserted through a surgical incision in the vessel wall. An alternative arrangement includes a delivery device on which the conduit is mounted, the device having a permanent or detachable incising element with a sharpened tip for penetrating the wall of the target vessel in conjunction with introducing the conduit. Another arrangement uses a sheath that restrains a collapsible conduit and is removed to deploy the conduit.

Additionally, the conduits of the invention are preferably, though not necessarily, placed with the portion in the myocardium spaced from the portion in the coronary vessel. That is, the channel passing through the myocardium is not beneath or immediately adjacent the vessel. Nonetheless, as shown above the conduit may be positioned transmurally in myocardial tissue directly or substantially beneath or adjacent the vessel. One benefit of the former method is that the conduit (or delivery device supporting the conduit) is introduced through the outer or anterior vessel wall to engage the lumen; it is not passed through the inner or posterior vessel wall, which tends to be more diseased than the outer wall.

It may be desirable to utilize a conduit delivery device having a portion surrounding the conduit to protect the conduit material prior to and during deployment. The device may have a bore that, in addition to receiving the aforementioned optional incising element so that may be extended and retracted, is configured to act as a flashback lumen and indicate when the device has entered a lumen containing blood, for example, a coronary artery or heart chamber. Of course, additional members, for example, a guide wire or guide catheter, may be used to deliver the conduit.

The conduits of the invention may be provided with a valve or other means for controlling or regulating blood flow. Suitable valves, as well as means for measuring myocardial thickness or verifying entry into the heart chamber, are disclosed in application Ser. No. 09/023,492, filed on Feb. 13, 1998, and entitled “Methods and Devices Providing Transmyocardial Blood Flow to the Arterial Vascular System of the Heart,” the entire subject matter of which has been incorporated herein by reference. Likewise, the conduits may be provided with a reservoir for retaining and discharging blood in a desired manner.

The conduits and delivery devices of the invention may be sized and configured differently from that specifically illustrated in the Figures. For instance, the cross-section of one or more portions of the conduit may be noncircular, e.g., elliptical to better match the profile of the target vessel. As a further example, the delivery device may be relatively short with the shaft assembly substantially rigid for use in an open-chest procedure. Alternatively, the device may be configured for use in either a minimally invasive or endovascular procedure, wherein the actuators for controlling the device components are located adjacent the proximal end of the device to allow remote deployment of the conduit, for example, as disclosed in the aforementioned, co-pending, commonly-owned application Ser. No. 09/304,140.

It will be appreciated that the features of the various preferred embodiments of the invention may be used together or separately, while the illustrated methods and devices may be modified or combined in whole or in part. As an example, either of the securing components could be formed as a multipiece or multilayer structure having a desired amount of rigidity or flexibility. Also, more than one conduit may be coupled to a manifold that is placed in communication with one source of blood so as to deliver blood to multiple target vessels. The conduits and devices of the invention may include removable or detachable components, could be formed as disposable instruments, reusable instruments capable of being sterilized, or comprise a combination of disposable and reusable components.

Further, it will be understood that the embodiments may be used in various types of procedures, for example, an open surgical procedure including a median sternotomy, a minimally invasive procedure utilizing one or more relatively small access openings or ports, or an endovascular procedure using peripheral access sites. Also, endoscopes or thoracoscopes may be used for visualization if the procedure is performed through very small ports. The different embodiments may be used in beating heart procedures, stopped-heart procedures utilizing cardiopulmonary bypass (CPB), or procedures during which the heart is intermittently stopped and started.

It will be recognized that the invention is not limited to the illustrated applications. For example, an inventive conduit may be coupled to an existing CABG graft that has partially or completely occluded over time by plugging the second conduit portion into the graft distal to the occlusion.

It will be recognized that the invention may be used to manufacture conduits the use of which is not limited to cardiovascular applications such as those illustrated and discussed above. For example, the invention may be used to produce conduits used to carry out many different bypass procedures, including, without limitation, femoral-femoral, femoral-popliteal, femoral-tibial, ilio-femoral, axillary-femoral, subclavian-femoral, aortic-bifemoral, aorto-iliac, aorto-profunda femoris and extra-anatomic. The conduit may be used to establish fluid communication with many different vessels, including, without limitation, the renal arteries, mesenteric vessel, inferior mesenteric artery, eroneal trunk, peroneal and tibial arteries. Still other applications for the invention include arteriovenous shunts. The conduit may have one, both or more ends configured to engage a target vessel for receiving blood from or delivering blood to another vessel.

The preferred embodiments of the invention are described above in detail for the purpose of setting forth a complete disclosure and for sake of explanation and clarity. It will be readily understood that the scope of the invention defined by the appended claims will encompass numerous changes and modifications.

What is claimed is:

1. A method for delivering blood from a heart chamber containing blood to a target vessel of a patient's vascular system, the method comprising steps of:
(a) providing a conduit having a lumen adapted to be placed in fluid communication with a heart chamber containing blood and an attachment portion adapted to be secured to a target vessel so as to communicate with the lumen of the target vessel;
(b) placing the conduit in fluid communication with the heart chamber;
(c) positioning a first component of the conduit attachment portion in the target vessel lumen adjacent a first area of the vessel wall, and positioning a second component of the conduit attachment portion adjacent another area of the wall;
(d) capturing the target vessel wall between the first and second components to secure the conduit to the target vessel; and
(e) delivering blood from the heart chamber into the conduit during at least one phase of the heart cycle.

2. The method of claim 1, wherein step (d) is performed by applying force to at least one of the first and second components to secure the conduit to the target vessel.

3. The method of claim 2, wherein step (d) is performed by introducing the first component into the target vessel lumen, positioning the second component against an exterior surface of the target vessel wall, moving the first component against an interior surface of the target vessel wall, and applying force to at least one of the first and second components to sandwich the target vessel wall.

4. The method of claim 3, wherein the first component is passed through an opening in the target vessel wall, and a sufficient amount of force is exerted against the one component to seal the opening in the target vessel wall and prevent blood leakage.

5. The method of claim 3, wherein the first component is passed through an opening in the target vessel wall, and the opening is the only penetration made in the target vessel wall.

6. The method of claim 5, wherein the first and second components contact the surfaces of the target vessel wall without penetrating the wall

7. The method of claim 1, wherein the conduit has a curved configuration to substantially match a curved target vessel wall.

8. The method of claim 1, wherein a portion of the conduit extends between the heart chamber and the target vessel and is disposed on an exterior of the patient's heart.

9. The method of claim 1, further comprising permitting the blood to flow from the conduit into the target vessel lumen in more than one direction.

10. A method for securing a conduit to a target vessel of a patient's vascular system, the method comprising steps of:
(a) providing a conduit having a lumen and an attachment portion adapted to be secured to a target vessel so that the conduit lumen is in fluid communication with the target vessel lumen, wherein the attachment portion includes first and second securing components;
(b) placing the first securing component at least partially with the target vessel lumen and positioning the first securing component adjacent one surface of the target vessel wall;
(c) placing the second securing component adjacent another surface of the target vessel wall; and
(d) applying a desired amount of force to the target vessel wall without the first or second component penetrating the tissue of the target vessel wall, thereby securing the conduit to the target vessel.

11. The method of claim 10, wherein step (d) is performed by applying the force by a mechanism selected from the group consisting of: springs, ratchets, screw threads, magnets, sutures and strings, clamps, clips, snaps, resilient bands or rings, and resilient conduit materials.

12. The method of claim 10, wherein the conduit is also placed in fluid communication with a source of blood selected from the group consisting of: the left ventricle, the right ventricle, the left atrium, the right atrium, the aorta, the pulmonary arteries, the pulmonary veins, coronary arteries, coronary veins, peripheral arteries, and peripheral veins.

13. The method of claim 12, wherein the target vessel is selected from the group consisting of: the aorta, the pulmonary arteries, the pulmonary veins, coronary arteries, coronary veins, peripheral arteries, and peripheral veins.

14. A method for securing a native artery to a target vessel of a patient's vascular system, the method comprising steps of:
(a) providing an attachment mechanism configured to be secured to a target vessel, the attachment mechanism having a lumen adapted to be placed in fluid communication with the target vessel lumen, wherein the attachment mechanism includes first and second securing components;
(b) placing one of the securing components at least partially within the lumen of the target vessel adjacent a surface of the target vessel wall;
(c) preparing the native artery to provide an exposed portion for securing the artery to the target vessel;
(d) coupling the other securing component to the exposed portion of the native artery and positioning the other securing component adjacent another surface of the target vessel wall; and
(e) sandwiching the target vessel wall between the securing components to secure the native artery to the target vessel.

15. The method of claim 14, wherein the native artery is one of the internal mammary arteries, and the exposed portion of the artery is a free end of the internal mammary artery.

16. A device for securing a conduit to a target vessel so that the conduit and the vessel are in fluid communication, the device comprising:
first and second securing components configured to engage different areas of the wall of a target vessel to provide a secure attachment, at least one of the components has a length and a width, the length being defined generally along the axis of the target vessel when the device is positioned in the target vessel;
wherein the length of the at least one securing component is greater than the width of the at least one securing component.

17. The device of claim 16, wherein the at least one securing component is generally rectangular.

18. The device of claim 17, wherein the at least one securing component has straight sides and at least one rounded end.

19. The device of claim 16, wherein the at least one securing is configured to engage an interior surface of the target vessel wall and comprises a base member with a coating of silicone.

20. The device of claim 16, wherein the other securing component is configured to overlie an exterior surface the target vessel wall and is saddle-shaped so as to substantially surrounds the at least one securing component.

21. A device for securing a conduit to a target vessel so that the conduit and the vessel are in fluid communication, the device comprising:
first and second securing components configured to engage different areas of the wall of a target vessel to provide a secure attachment, at least one of the components has a length and a width, the length being defined generally along the axis of the target vessel when the device is positioned in the target vessel;
wherein one of the first and second securing components is configured to directly engage an opening in the wall of a target vessel and is sized for a particular size range of target vessels; and
wherein the one securing component has an outlet with a cross-sectional area of at least 50% of the cross-sectional area of the target vessels in the particular size range.

22. The device of claim 21, wherein the one securing component has an outlet with a cross-sectional area of at least 70-80% of the cross-sectional area of the target vessels in the particular size range.
